(19) United States
(12) Patent Application Publication
(10) Pub. No.: US 2021/0373014 A1
(43) Pub. Date: Dec. 2, 2021

(54) AN INTEGRATED MICROFLUIDIC ELECTRODE ARRAY SYSTEM FOR ENZYME-LINKED IMMUNO-SORBENT ASSAY (EASY-ELISA) FOR POINT-OF-CARE DETECTION OF MOLECULAR AND CELLULAR BIOMARKERS

(71) Applicants: THE GENERAL HOSPITAL CORPORATION, Boston, MA (US); MASSACHUSETTS INSTITUTE OF TECHNOLOGY, Boston, MA (US)

(72) Inventors: Galit Alter, Winchester, MA (US); Aniruddh Sarkar, Boston, MA (US); Jongyoon Han, Boston, MA (US)

(21) Appl. No.: 16/758,731

(22) PCT Filed: Oct. 23, 2018
(86) PCT No.: PCT/US2018/057166

§ 371 Date: Apr. 23, 2020

Related U.S. Application Data
(60) Provisional application No. 62/575,944, filed on Oct. 23, 2017.

(51) Int. Cl. G01N 33/569; G01N 33/543; B01L 3/00

(52) U.S. Cl. CPC G01N 33/5695; G01N 33/5438; G01N 2333/35; B01L 2300/0883; B01L 2300/0645; B01L 3/502715

(57) ABSTRACT

A method for detection of antibodies in a biological sample. The method includes steps of: immobilizing antigens specific to the antibodies between at least two electrodes; binding the antibodies from the biological sample to the antigens; binding probes linked with an enzyme to the antibodies; exposing the enzyme to a metal substrate; depositing a metal layer based on exposing the enzyme to the metal substrate; measuring an electrical property of the metal layer between a first electrode of the at least two electrodes and a second electrode of the at least two electrodes; and detecting, based on measuring the electrical property of the metal layer, the antibodies in the biological sample.

### CROSS-REFERENCE TO RELATED APPLICATIONS

This application claims the benefit of U.S. Provisional Patent Application Ser. No. 62/575,944 filed on Oct. 23, 2017 and entitled “An Integrated Microfluidic Electrode Array System for Enzyme-Linked Immuno-Sorbent Assay for Point-of-Care Detection of Molecular and Cellular Biomarkers.”

### STATEMENT REGARDING FEDERALLY SPONSORED RESEARCH

This invention was made with government support under AI109755 awarded by the National Institutes of Health. The government has certain rights in the invention.

### FIELD OF THE INVENTION

This document relates to apparatus and methods for detecting targets in a biological sample based on an ELISA-type assay using an electrical-based detection scheme and a microfluidic sample handling apparatus.

### BACKGROUND

Assays based on specific interaction and binding of biomolecules find wide use across biology and in clinical diagnostics for a range of diseases, the most common example being immunoassays which measure the presence or concentration of a molecule in biological fluids via its specific binding to an antibody. A commonly used method for binding-based assays is the enzyme-linked immunosorbent assay (ELISA) in which the binding of the target analyte from the sample to a specific capture agent is amplified and measured via a coupled secondary enzymatic reaction, which generates a colored product whose concentration is measured, most commonly, via optical absorbance. Multiple dilutions of the sample and a reference standard are usually analyzed to fit binding curves and quantitate analyte concentration or titer or obtain other parameters such as binding affinity (e.g. dissociation constant K_{D}). ELISAs offer highly sensitive detection and accurate quantitation and are considered the gold standard in detection of many clinical biomarkers. However, ELISAs often require expensive instrumentation and expertise and hence are often restricted to being performed in a clinical or research laboratory environment.

### SUMMARY OF THE PRESENT DISCLOSURE

Accordingly, in various embodiments the present invention provides methods, apparatus, and systems for one or more of: performing direct electrical impedance-based detection and quantitation of sensitive enzymatically-amplified binding-based bioassays in an inexpensive portable platform without the use of any intermediate optics, light sources, or optical detectors; electrical detection and quantitation of molecular biomarkers such as RNA, DNA, proteins (e.g. antigen-specific antibodies), or specific protein modifications (e.g. glycoforms of antigen-specific antibodies) in serum, blood or other bio-fluids; electrical detection and quantitation of cellular biomarkers and abundance or counts of specific cell types or cells with specific surface, cytosolic, or secreted markers or ratios of abundance of these cells in blood or other bio-fluids; sensitive electrical detection of molecular and cellular biomarkers which may be achieved by directly converting analyte binding with specific detection probes to an electrical impedance signal by probe-directed enzymatically-amplified deposition of metal nanoparticles on a microelectrode array chip, enabling flow of electrical current and its increase with analyte concentration; and/or integrated microfluidic serial dilution or distribution of sample, enabling quantitation via titer or concentration measurement or digital counting-based assays.

In one or more example embodiments of the present disclosure, a method is provided for detection of antibodies in a biological sample. The method includes steps of: immobilizing antigens specific to the antibodies between at least two electrodes; binding the antibodies from the biological sample to the antigens; binding probes linked with an enzyme to the antibodies; exposing the enzyme to a metal substrate; depositing a metal layer based on exposing the enzyme to the metal substrate; measuring an electrical property of the metal layer between a first electrode of the at least two electrodes and a second electrode of the at least two electrodes; and detecting, based on measuring the electrical property of the metal layer, the antibodies in the biological sample.

In one or more example embodiments of the present disclosure, a method is provided for detection of a target in a biological sample, the method including the steps of: immobilizing antibodies specific to the target between at least two electrodes; binding the target from the biological sample to the antibodies; binding probes linked with an enzyme to the target; exposing the enzyme to a metal substrate; depositing a metal layer based on exposing the enzyme to the metal substrate; measuring an electrical property of the metal layer between a first electrode of the at least two electrodes and a second electrode of the at least two electrodes; and detecting, based on measuring the electrical property of the metal layer, the target in the biological sample.

In one or more example embodiments of the present disclosure, a serial auto-dilution device is provided including: a first inlet for a biological sample; a first channel connected to the first inlet, the first channel including a plurality of chambers; a second channel connected to a source of a dilution buffer; a first plurality of connection channels connecting the second channel to the first channel between each of the respective plurality of chambers; a third channel connected to an outlet; and a second plurality of connection channels connecting the first channel to the third channel between each of the respective plurality of chambers, each of the first channel, the second channel, the third channel, the first plurality of channels, and the second plurality of channels being configured such that the biological sample flows through the first channel and the dilution buffer flows through the second channel and the first plurality of channels to produce increasingly diluted mixtures of biological sample and dilution buffer in each of the plurality of chambers.

In one or more example embodiments of the present disclosure, a microfluidic serial dilution apparatus is provided, including: a substrate including: a sample input opening coupled to a sample channel, a buffer input opening coupled to a buffer channel, a first sample chamber coupled to the sample channel, a second sample chamber coupled to the first sample chamber by the sample channel, a first side channel coupling the buffer channel to the sample channel between the first sample chamber and the second sample chamber, the first side channel having a first resistance, and a second side channel coupling the sample channel to a waste channel between the first sample chamber and the second sample chamber, the second side channel having a second resistance, addition of a sample to the sample input opening and a buffer to the buffer input opening causing a first sample fluid to be in the first sample chamber and a second sample fluid to be in the second sample chamber, the second sample fluid having a lower concentration of sample than the first sample fluid.

In one or more example embodiments of the present disclosure, a method for treating a disease or condition in a subject is provided, the method including: assaying a sample obtained from the subject to determine an antibody glycosylation state, the antibody glycosylation state being indicative of the disease or condition; and administering a treatment for the disease or condition if the antibody glycosylation state is indicative of the presence of the disease or condition.

In one or more example embodiments of the present disclosure, a method for diagnosing a disease or condition in a subject, the method including: assaying a sample obtained from the subject to determine an antibody glycosylation state, the antibody glycosylation state being indicative of the disease or condition; and diagnosing the disease or condition in the subject based on the presence of an antibody glycosylation state indicative of the disease or condition.

The foregoing and other aspects and advantages of the present disclosure will appear from the following description. In the description, reference is made to the accompanying drawings that form a part hereof, and in which there is shown by way of illustration one or more exemplary versions. These versions do not necessarily represent the full scope of the invention.

### BRIEF DESCRIPTION OF THE DRAWINGS

The following drawings are provided to help illustrate various features of example embodiments of the disclosure, and are not intended to limit the scope of the disclosure or exclude alternative implementations.

FIG. 1A shows the assay principle for electrical measurement of antigen-specific antibody titer using an anti-IgG probe and glycosylation using a lectin probe followed by enzymatic silver metallization on a gold microelectrode array.

FIG. 1B shows the assay principle for electrical detection of cells with specific surface or cytosolic markers by using antibodies to capture them on a gold microelectrode array and binding of a detection antibody probe followed by enzymatic silver metallization.

FIG. 1C shows the assay principle for electrical detection of cells with specific secreted markers by single cell isolation in nanoliter-scale chambers and capture of secreted markers using antibodies on a gold microelectrode array and binding of a detection antibody probe followed by enzymatic silver metallization.

FIG. 2A shows a schematic of a unit module for an m-fold dilution of sample with buffer in an n chamber dilution series.

FIG. 2B shows a schematic of a microfluidic serial dilution network using a series of m-fold dilution unit modules resulting in n-chamber dilution series for titer measurement.

FIG. 2C shows a schematic of a microfluidic sample distribution network for isolation of sample into a large number of separate nanoliter or picoliter-scale chambers and digital detection using an ON/OFF signal from each chamber and counting.

FIG. 3 shows a photolithography mask design for implementing a Microfluidic Electrode Array System for Enzyme-Linked Immuno-Sorbent Assay (EASy-ELISA) by integrating gold microelectrode arrays inside the eight assay chambers of a network implementing a 2-fold dilution series, where two separate assays (e.g. antigen-specific antibody titer and glycosylation) can be performed on a single chip by division of sample.

FIGS. 4A-4C provide a demonstration of electrical detection using streptavidin-HRP binding on immobilized biotinylated bovine serum albumin. FIGS. 4A and 4B show optical micrographs of microelectrode arrays and FIG. 4C shows impedance spectra of electrodes with negative controls (with enzyme) and positive controls (BSA only).

FIGS. 5A-5C show a microfluidic serial dilutor: FIG. 5A shows design and simulation results; FIG. 5B provides fluorescence micrographs of a first stage showing dilution of a sample containing a FITC-tagged protein; and FIG. 5C shows a graph depicting quantification of a logarithmic dilution series obtained experimentally and graphed in comparison with results predicted by a simulation.

FIG. 6A shows an embodiment of an integrated electrical enzyme-linked immunosorbent assay chip with a serial dilutor bonded on top of the electrode array, where two dilutors are fabricated in parallel for titer and glycan measurements; FIG. 6B shows variation of impedance (at f=2 kHz) as a function of enzyme dilution, where metallization reaction time can be used to tune the response to digital or analog regimes; and FIG. 6C shows an inset from FIG. 6B depicting the limit of detection estimation.

FIGS. 7A-7C show results of a partial least squares discriminant analysis of impedance signatures obtained above results in accurate discrimination of 4 LTBI and 6 ATB samples (AUC=1). Glycan binding signatures obtained using SNA on CFP10-specific and LAM-specific antibodies have the highest loading followed by LAM, PPD, and CFP10-specific titers.

FIG. 8A shows a diagram of an embodiment of a smartphone-based inexpensive POC ELISA system using the EASy-ELISA technology disclosed herein.

FIG. 8B shows a photograph of an embodiment of a cellphone-based device for implementing EASy-ELISA detection.

FIG. 9 shows graphs of dual dilution curves which distinguish differences in titer and lectin binding via slopes of the curves.

FIG. 10 shows dual dilution curves as in FIG. 9 using MTB PPD (left) or MTB ESAT6 (right) and lectins SNA (top) or RCA1 (bottom).

FIG. 11 shows dual dilution curves for non-MTB antigens tetanus (left) and pneumococcus (right) and lectins SNA (top) or RCA1 (bottom).

FIGS. 12-15 show antigen-specific lectin-binding signatures in TB in which optimization of antigen choice can provide improved diagnostic power. Data are shown for antigens PPD (FIG. 12), Ag85A (FIG. 13), ESAT6 (FIG. 14), and CFP10 (FIG. 15).

FIG. 16 shows analysis of pediatric TB samples using lectins SNA (left) or RCA1 (right).

FIG. 17 shows analysis of typhoid samples using hemolysin E-specific IgG antibodies.

### DETAILED DESCRIPTION OF THE PRESENT DISCLOSURE

For clinical diagnosis, there has long been a need to perform assays rapidly, inexpensively, and in a relatively non-invasive manner (e.g. from a drop of blood obtained from a finger-prick) at a point-of-care (POC), while still maintaining accuracy. In the context of infectious diseases (e.g. Tuberculosis, HIV/AIDS), which have a high burden in resource-limited settings, accurate POC tests are considered critical to disease control and eradication. Even in relatively resource-rich settings, inexpensive POC tests can play a key role in reducing health care costs and improving access and outcomes.

Since their inception, microfluidics and lab-on-chip platforms have held the promise of offering the combination of low sample use, portability, automation, and low cost required for POC diagnostics via miniaturization. One of the bottlenecks in miniaturizing and directly porting clinical diagnostic assays to microfluidic POC platforms has been that optical absorption, commonly used for detection in ELISAs and other diagnostic assays, scales unfavorably with reduction in path length (e.g. Transmittance, T˜e^{-E.L.C.} where E, L, C are absorptivity, path length, and concentration, respectively) and is thus unsuitable for sensitive yet inexpensive micro-scale detection. In general, more sensitive optical techniques (e.g. laser-induced fluorescence) can be complex and expensive to implement in a portable instrument. Some cellphone-based optical methods have been recently developed, however they generally offer lower sensitivity than macro-scale systems. Another difficulty encountered in miniaturization of clinical diagnostics is performing microscale sample handling and preparation without using bulky, complex, and expensive off-chip valves, pumps, and robotics, the use of which defeats the very purpose of miniaturization of the assay itself.

One possible alternative would be to develop a system based on existing commercial lateral-flow based binding assays or ‘dipstick tests,’ which are widely used as POC or home-use diagnostics (e.g. pregnancy test kits). These use capillary wicking in a porous support membrane to drive flow of sample and antibodies labeled with gold nanoparticles to provide a binary (i.e. ‘yes/no’) color signal visible to the naked eye. However, while these are simple to use and affordable, they are often not quantitative and are usually much less sensitive than ELISAs.

Microfluidic adaptations of these assays have used gold nanoparticle labels as catalysts for silver deposition to generate an amplified optical signal detectable using portable optical detection methodologies. Such silver enhancement has also been used in nanoparticle based detection of DNA and other molecules. These assays, however, do not offer either the sensitivity or full functionality of traditional ELISAs, instead performing only single-point measurements and offering only binary results, or using complex off-chip optics and fluidics, and hence remain relatively expensive and bulky. Thus a compelling advantage, in terms of cost and benefits, of microfluidic ELISA systems which can drive their widespread adoption in POC diagnostics has remained elusive.

Accordingly, disclosed herein are embodiments of a miniaturized, sensitive, and direct electrical detection and quantitation scheme for binding-based assays using probe-directed enzymatic metallization on a microelectrode array and a microfluidic nanoliter-scale sample handling and distribution network and integrate these to build a single-chip, point-of-care diagnostic platform for molecular and cellular biomarkers which is referred to as the Electrode Array System for Enzyme-Linked Immuno-sorbent Assay (EASy-ELISA). This chip can be directly interfaced with portable, battery-powered electronics to build an inexpensive POC, ELISA-based sensitive and quantitative diagnostics platform without the use of any intermediate optics, light sources, or optical detectors or any off-chip pumps, valves, or robotics. The use of EASy ELISA is demonstrated here for POC-based diagnosis and stratification of Tuberculosis (TB) into latent TB infection (LTBI) and active TB (ATB) using novel antigen-specific antibody glycosylation biomarkers. The principles underlying the components of the EASy-ELISA chip are described below. In various embodiments, the chip may include an interdigitated microelectrode array detector as well as a microfluidic handling system that automatically generates serial dilutions of a sample without requiring an active pumping mechanism.

Using a scheme such as those disclosed herein, detection of antibodies or other targets (e.g. cells or proteins) from a biological sample is indicative of at least one of a disease state or a presence or activity of an infectious agent. The biological sample may include a bodily fluid, which in various embodiments can include at least one of blood, sputum, urine, saliva, or cerebrospinal fluid. In various embodiments, the infectious agent may be tuberculosis (TB), where antigens to detect TB may include one or more of PPD, LAM, CFP10, ESAT6, or Ag85A.

Electrical Detection

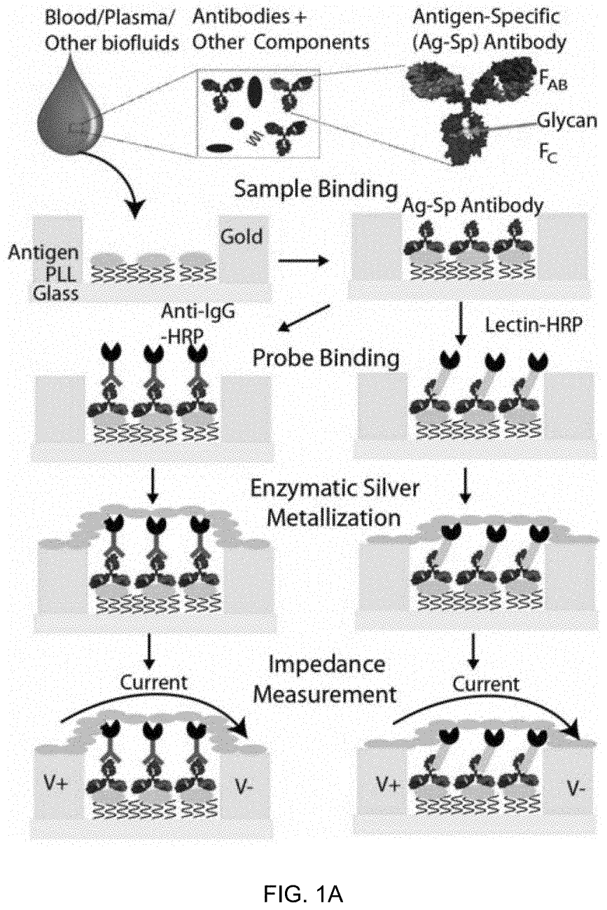

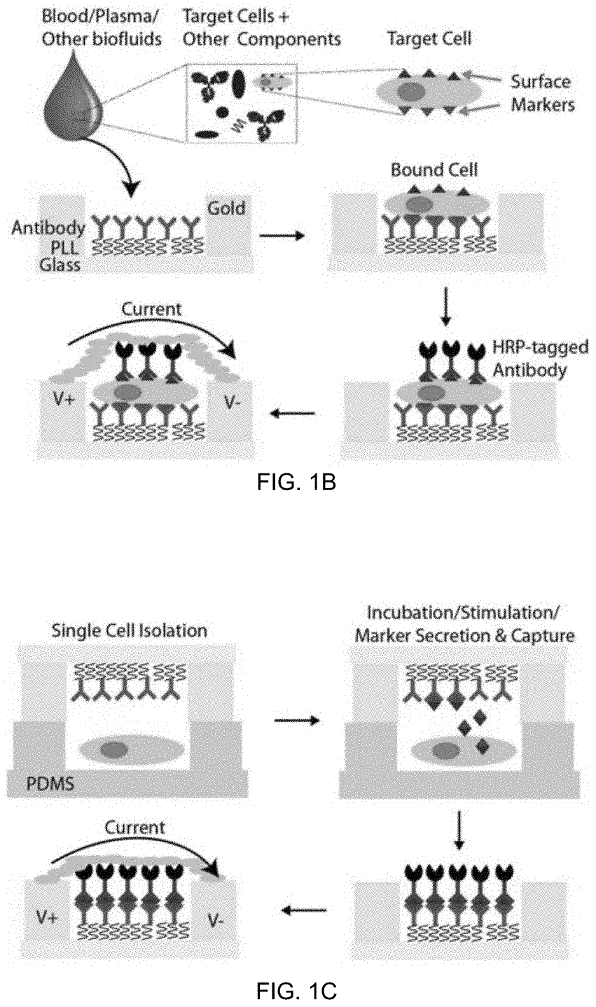

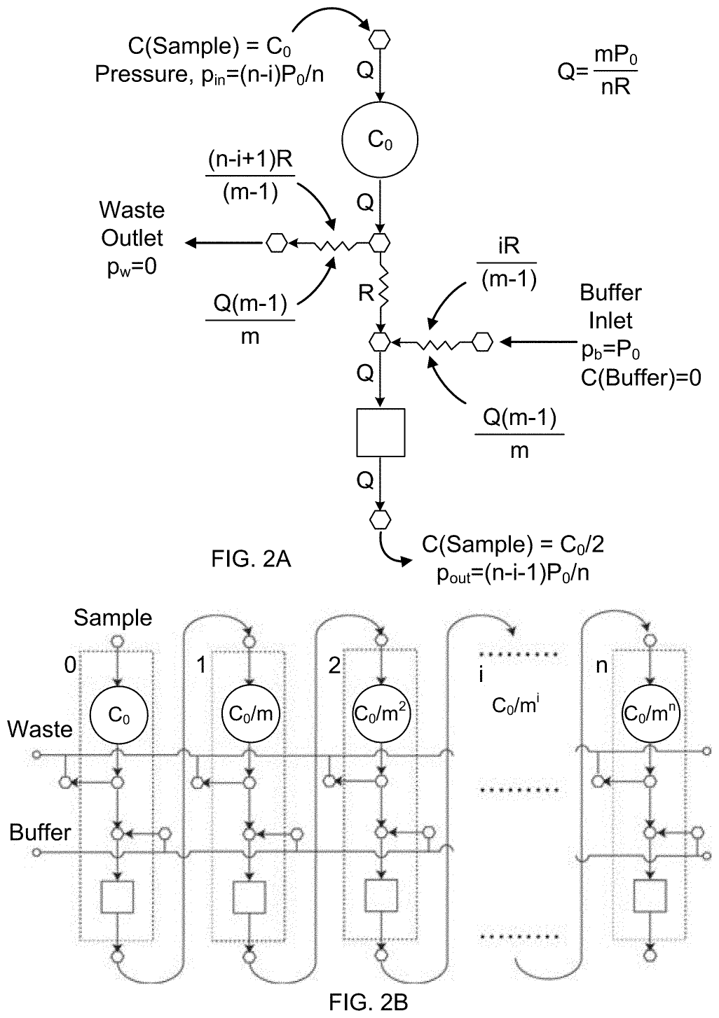

Several different embodiments of assay schemes for electrical detection of molecular and cellular biomarkers are shown in FIGS. 1A-1C, which show diagrams for assay schemes, including schemes to: (i) detect antigen-specific antibodies as well as for detecting subpopulations of those antibodies that are glycosylated (i.e. to indicate the glycosylation state of the antibody subpopulation, FIG. 1A); (ii) detect cells and/or particular cell-surface markers (FIG. 1B); and (iii) detect secreted cellular markers released from cell(s) into solution. Other variations on the basic principles disclosed herein are also possible.

In one scheme that is outlined in FIG. 1A, antigen-specific antibodies may be captured from patient serum using antigens immobilized (e.g. using poly-L-lysine, PLL) on a gold interdigitated microelectrode array. Anti-human immunoglobulin (IgG-HRP) or lectins labeled with the enzyme horseradish peroxidase (lectin-HRP) are then used as probes to detect all antibodies as well as a subset of antibodies modified with glycans, respectively. Addition of a silver substrate to the samples results in HRP-catalyzed deposition of a layer of silver, which enables the flow of electrical current between the microelectrodes and hence can be electrically detected by a change in an electrical property of the microelectrode array such as impedance. This scheme can be generalized and adapted to provide POC electrical detection of other proteins (e.g. pathogen-specific proteins) or other modifications of proteins (e.g. phosphoforms) via the use of appropriate capture agents and detection probes. In various embodiments, other enzymes (e.g. alkaline phosphatase, beta-galactosidase) may be used instead of or in addition to HRP and other metals (e.g. gold, platinum) may be used instead of or in addition to silver.

This scheme can also be adapted to provide electrical detection of specific nucleic acid sequences (DNA or RNA), including pathogen or host markers, and can enable PCR-free POC nucleic acid detection. These schemes can further be adapted to electrically detect and quantitate specific cells with particular cell-surface, cytosolic, or secreted cellular markers via the use of the appropriate capture agents and detection probes in combination with associated fluidics; in some embodiments, such as the detection of secreted markers, a microfluidic system may help confine the sample to allow detection without dilution of the sample (e.g. due to diffusion). This is shown in the schematics in FIGS. 1B and 1C. In other embodiments in which the target includes one or more cells, the cells may be permeabilized (e.g. using detergent) to provide access to internal antigens within the cells. In still other embodiments, the cells may be incubated for a period of time to permit secretion of antigens.

In the scheme outlined in FIG. 1B, antibodies that are specific for a particular target (e.g. a cell and/or protein) are immobilized on a substrate. A biological sample containing the target cells and/or proteins is then added to the antibody-containing substrate to bind the target to the antibodies. Next, a probe that is specific for the target cells/proteins is added to the system, where the probe has an enzyme such as horseradish peroxidase (HRP) coupled to it. The HRP-coupled probe (e.g. an HRP-tagged antibody) is then exposed to a metal substrate (e.g. a solution containing silver) and the enzyme then catalyzes deposition of a layer of the metal in the vicinity of the sample. As this is being performed between a pair of electrodes (e.g. which may be part of a microelectrode array), the deposited metal layer may change an electrical property between the pair of electrodes, e.g. change the resistance or impedance. The measurements of the electrical property and in particular to the changes in the electrical property of the electrode following this procedure then allows one to detect the presence or absence of the target cell and/or protein. Detection of the target may also include quantitation of levels of the target, particularly when the particular sample is part of a serial dilution of the biological sample, as discussed further below.

In the scheme outlined in FIG. 1C, one or more cells may be isolated within a small space such that any materials secreted from the cell(s) is able to contact antibodies or other probes that are specific for the secreted materials. Following exposure to the fluid containing secreted materials, the antibodies or other probes with secreted materials attached thereto are processed in a manner as described above for the scheme of FIG. 1B in which the sample is exposed to a secondary probe (e.g. an antibody) with an enzyme attached thereto, followed by deposition of a metal layer and measurement of an electrical property of the electrodes.

Other electrical detection methods for ELISAs usually rely on more complex electrochemical techniques (e.g. pulse voltammetry) which use external stable reference electrodes and instrumentation such as a potentiostat. On the other hand, the electrical detection technique described above, which uses measurement of an electrical property such as electrode resistance/impedance, can be performed with a simple handheld multimeter or using single-chip integrated circuits for performing such resistance or impedance measurements.

Microfluidic Sample Dilution and Distribution

Microfluidic sample handling and distribution can facilitate inexpensive automated quantification of molecular and cellular biomarkers in conjunction with the above electrical detection scheme. Specifically, two different modes of quantification that can be enabled by different microfluidic sample processing modules are exemplified here.

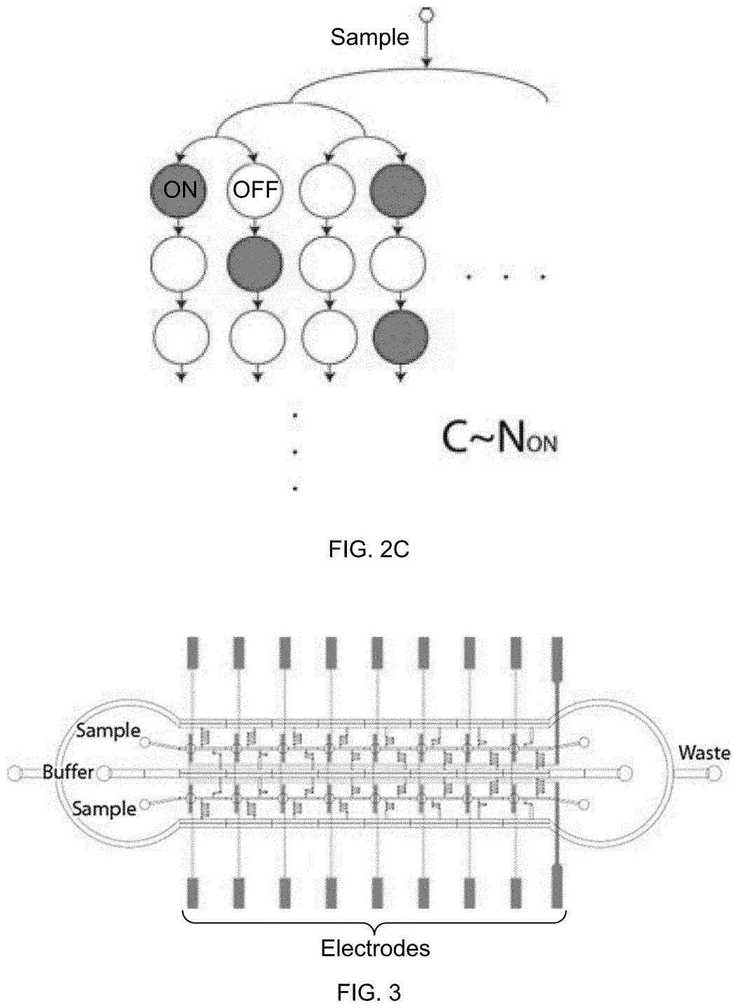

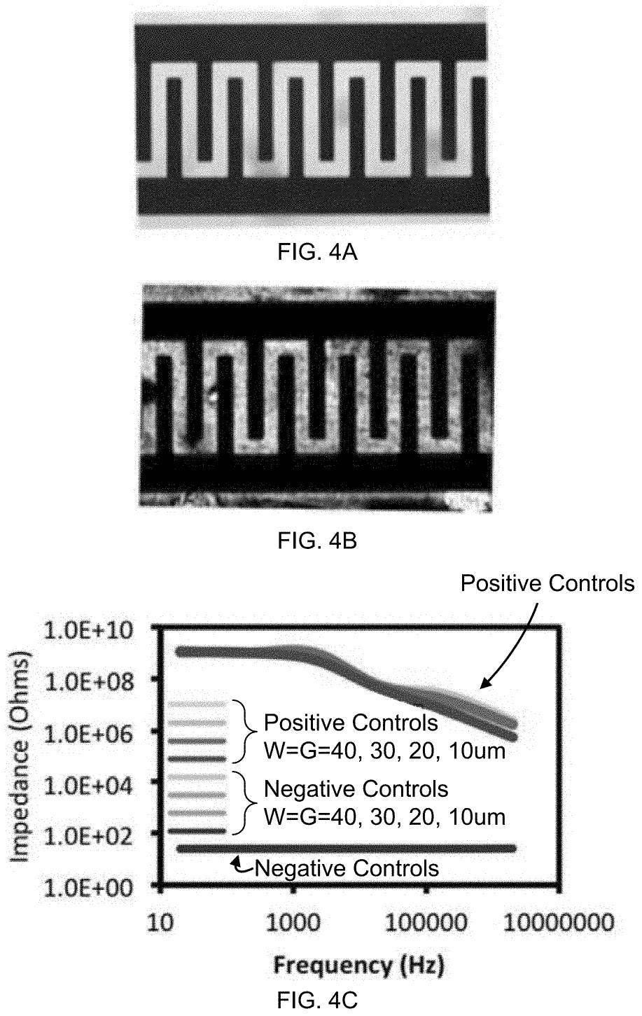

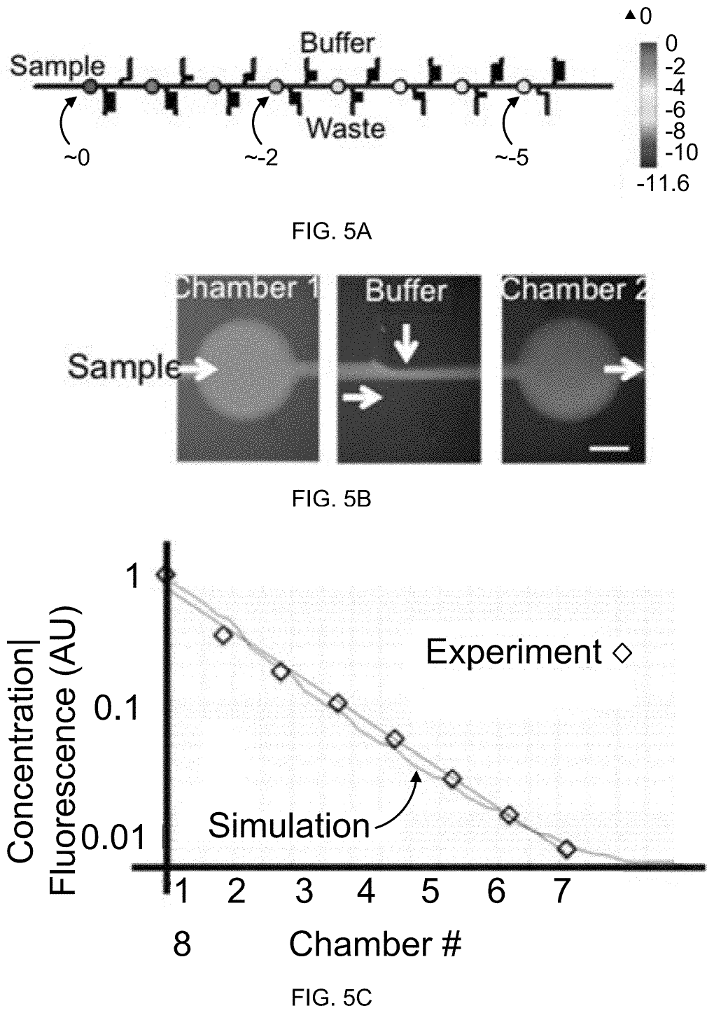

First, for relatively high abundance molecular or cellular markers, titer measurements can be performed by serially diluting the sample with an appropriate dilution buffer and measuring the highest dilution at which the marker is still detectable. Currently, titer measurements are performed using micropipettes and microtiter plates, either manually by trained laboratory technicians or automatically by programmed sample handling robots. This can be expensive and fluid handling performed this way is usually done using sample volumes at the microliter scale or above. Accordingly, disclosed herein is a simple and inexpensive yet automated and extremely sample-efficient microfluidic dilution scheme, which can dilute nanoliter scale samples repeatedly to generate a logarithmic dilution series using gravity- or pressure-driven flows from single sample and buffer inputs. FIG. 2A shows an equivalent circuit diagram of a unit microfluidic dilution module which performs an m-fold dilution by mixing the sample and buffer flows at a m:l ratio, which is achieved by choosing appropriate flow resistances of the buffer and waste channels. This module can be linked into an n unit network that enables a logarithmic dilution series (1, 1/m, 1/m², . . . , 1/mⁿ) as shown schematically in FIG. 2B. Notably, the whole network may be driven by as few as two inputs (namely, sample and buffer inlets) and does not require any additional manual or automated pipetting. In contrast, other microfluidic dilution or concentration gradient generation devices are generally very complex and typically require complex pneumatic controls.

Second, for very low abundance molecular markers or for cells and cellular markers, ‘digital’ or counting assays can be performed. Here the sample may be divided by the serial dilution device into separate chambers, where each separate chamber is evaluated as being ‘ON’ or ‘OFF’ for the presence or absence of the target marker, respectively, and where the number of ON chambers is counted to estimate the marker concentration (e.g. using Poisson statistics). With an appropriately small chamber size (e.g. in the nanoliter (nL) or picoliter (pL) range), even single molecules or single cells can be detected and counted. An embodiment of a microfluidic network that enables this is shown schematically in FIG. 2C.

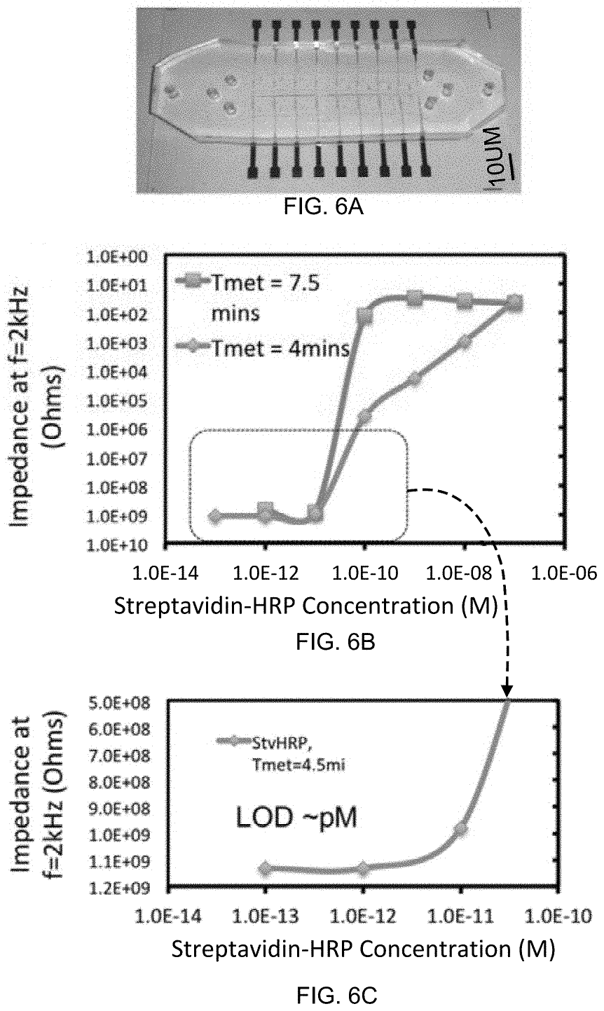

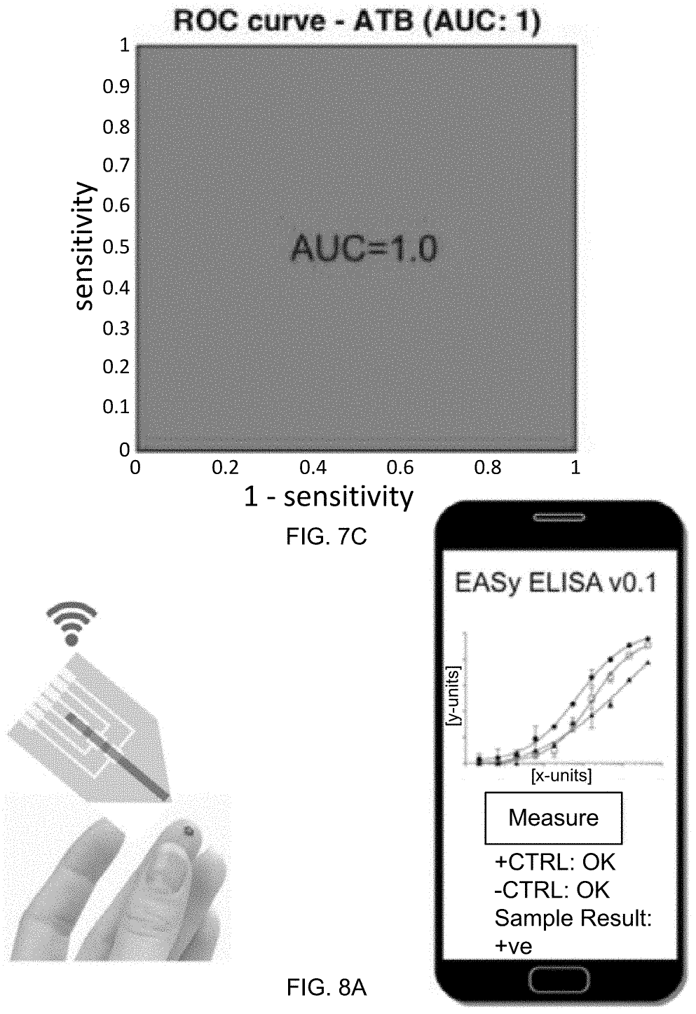

In general, the sample may be diluted at each stage by combining with buffer, while excess sample is diverted to a waste channel. The relative amounts of sample and buffer that are combined at each stage is controlled by changing the relative resistance of the inflow of buffer and outflow of waste. One manner in which resistance may be changed in a controlled manner is to change the lengths of the side channels, as shown in FIGS. 3 and 5A. In some embodiments, the change in resistance may be effected by including various numbers of bends or ‘switchbacks’ in the channels in order to increase the length of the channel, while still containing the channel within a limited region of the chip. As seen in FIG. 3, between the first and second stages, the channel leading from the main sample channel to the waste channel (in the upward direction in FIG. 3) includes numerous switchbacks, effectively increasing the length and hence the resistance of this channel, whereas the channel leading from the buffer channel to the sample channel includes only a single bend, giving this channel a shorter effective length and hence a lower resistance. The effect of this combination of side channels is to provide a relatively high amount of dilution of the sample between the first and second stages. At each subsequent stage the relative amounts of buffer and (diluted) sample that are combined is reduced by increasing the effective length/resistance of the buffer channel (decreasing the amount of buffer that enters the sample channel) and decreasing the effective length/resistance of the waste channel (increasing the amount of sample/diluted sample that exits the sample channel into the waste channel). Given that the sample at the beginning of each stage has been diluted from the original sample or previous stage, less and less buffer may be needed to attain a particular concentration or dilution level compared to the amount required in the earlier stages. Although in the examples presented the resistance is adjusted by changing the lengths of the side channels, in various embodiments other channel parameters instead of, or in addition to, length (L) may be adjusted, including the channels' width (w) and/or height (h) (where changes in either or both parameters change the channels' cross-sectional area), in order to change the resistance (R_{flow}) of a microfluidic flow channel having a rectangular cross-sectional shape according to the following formula:

$${R}_{flow} = \frac{12 η L}{( 1 - 0.63 ( \frac{h}{w} ) )} \cdot \frac{1}{{wh}^{3}}$$

where η is fluid viscosity.

In various embodiments, fluid flow through the serial dilution system may simply be driven by gravity, which is simple and cost-effective and lends itself to producing a low-cost POC device. Nevertheless, in various embodiments an active pumping mechanism may be included and in fact may be seamlessly incorporated into the devices disclosed herein. In certain embodiments, the inclusion of an active pumping mechanism (to drive one or both of the sample and/or the buffer flows) would provide a finer degree of control over flow rates without increasing the volume of sample that is needed and can also provide constant and robust flow rates regardless of the orientation of the device relative to gravity.

Integrated Assay and Multiplexed Detection

The microfluidic sample-handling networks and the electrical detection scheme described above can be integrated by simply enclosing microelectrode arrays which include immobilized capture agent (e.g. antigen or antibodies) within the assay chambers in the dilution network. A specific example of this is shown in the photolithography mask design in FIG. 3, in which microelectrode arrays have been integrated inside a dilution network that performs eight serial 2-fold dilutions (n=8, m=2) from single sample and buffer inputs. As shown in FIG. 3, a second sample input can be included so as to provide a second set of eight serial dilutions. As the first and second inputs are separated from one another by the buffer channel, two different samples can be added to the two sample inputs. Alternatively, the same sample may be loaded into each sample input but different targets may be detected in each set of serial dilutions, as discussed below.

Multiplexing or simultaneous detection of different analytes from a small volume of a single sample can be achieved in one of two ways:

A. As shown in FIG. 3, the sample can be divided into parallel dilution networks, each having electrodes with separate capture agents immobilized on them. As each of the microfluidic dilution networks uses only a few nanoliters of sample, many different analytes may be detected using limited sample volumes. This scheme allows for use of separate probes for the different analytes in the separate channels (e.g. anti-IgG and lectins) as the detection electrode arrays used to detect them can be physically isolated in different microfluidic chambers for the probe binding step.

B. A sample-efficient multiplexing scheme can be implemented by integrating multiple microelectrode arrays with different immobilized capture agents inside each assay chamber of a dilution network. As the silver deposition occurs locally on the surface of each microelectrode array, multiple targets can be detected simultaneously without crosstalk.

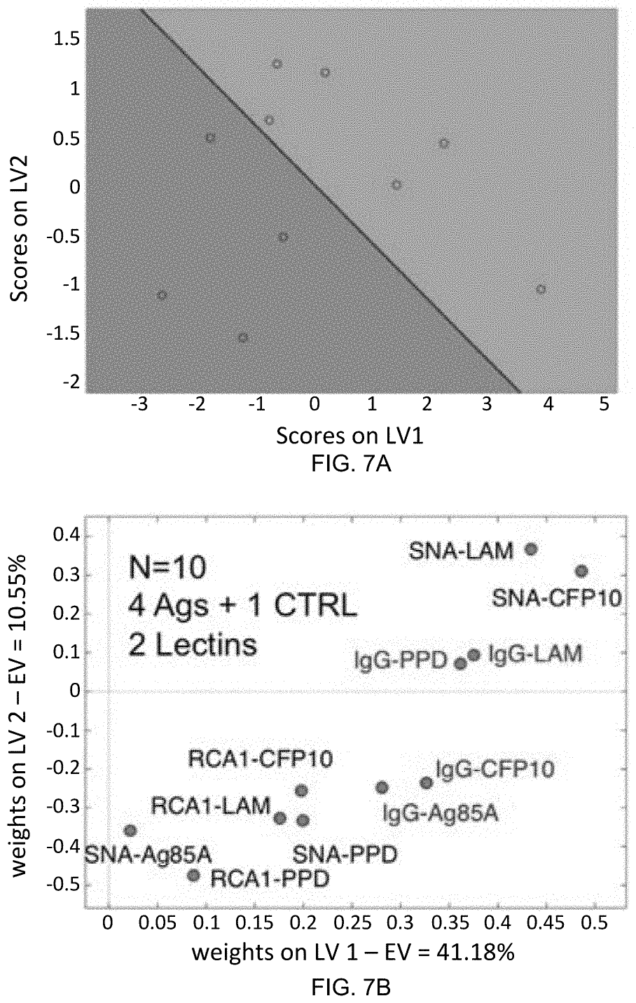

In some embodiments, interdigitated microelectrode arrays (FIG. 4A) were fabricated on glass substrates using standard microfabrication processes, which involved photolithography and electron-beam deposition of thin titanium (10 nm) and gold (100 nm) films followed by metal lift-off. Electrode arrays having a number of different electrode width and inter-electrode gap (w & g=10 μm, 20 μm, 30 μm, 40 μm) parameters were fabricated. The electrical detection scheme was tested via binding and detection of a streptavidin-HRP conjugate (100 μg/mL) as a target analyte using a biotinylated bovine serum albumin (BSA) as a capture layer immobilized on the glass substrate using a poly-L-lysine intermediate layer. After washing away the excess unbound targets, the silver substrate solution (EnzMet™, Nanoprobes Inc.) was added to the electrode array and the metallization reaction was observed under a microscope (FIG. 4B), where the reaction was allowed to proceed for a fixed amount of time (t=8 min). Electrodes were then washed and dried and their impedance was measured using an Agilent E4980A precision LCR meter. Measuring the electrical impedance spectra of the electrodes revealed more than seven orders of magnitude of change in impedance, from an “OFF” state or negative control with no target analyte at one end of the range, to an “ON” state or positive control with target analyte bound at the other end of the range (FIG. 4C). This wide range indicates the high sensitivity and dynamic range that this scheme can achieve in the detection of biomolecules and cells.

The negative control electrodes display a characteristic ‘open-circuit’ or capacitive impedance spectrum (negative controls shown as overlapping horizontal straight lines just below the “1.0E+02” level in FIG. 4C) while the positive control electrodes display a short-circuit' or resistive impedance spectrum (positive controls shown as a series of traces near the top of the graph in FIG. 4C). These results indicate that, in certain embodiments, a single frequency AC or even a static (DC) measurement may be used for this assay. In keeping with this proposed simplified detection scheme, a single frequency (f=2 kHz) was used here for subsequent measurements. Further, no significant difference was observed in the impedance spectra when using interdigitated microelectrode arrays with electrodes having different widths and gaps. This indicates that even inexpensively fabricated, relatively large electrodes (e.g. made via screen printing—w,g˜100 μm or above) can be used to further reduce the cost of this detection method. Accordingly, electrodes with w=g=40 μm were used here for further measurements.

Next, a microfluidic serial dilutor network was designed based on the scheme shown in FIGS. 2A and 2B. In one embodiment, the microfluidic serial dilutor network can generate an 8-point, 2-fold dilution series (n=8, m=2) automatically from single sample and buffer inlets/inputs by repeated automatic mixing at pre-programmed ratios using gravity-driven flow without any further manual pipetting. In various other embodiments, specific dimensions of sample, buffer, and waste arms of each unit module and a diffusion-based mixer may be designed using suitable software, for example using a coupled COMSOL simulation of fluid flow and solute transport of antibodies as model molecules. The results for such a simulation are shown in FIG. 5A. The serial dilution network was fabricated in a substrate made of PDMS using standard soft lithography methods and its operation was tested using a suspension of fluorescently labeled protein (10 ug/ml of FITC-tagged IgG in 1× PBS) as sample and 1× PBS as the dilution buffer. Micrographs of the first two assay chambers and buffer flow entrance are shown in FIG. 5B, with a noticeable decrease in brightness (=decreased concentration) being seen from the first assay chamber (FIG. 5B, left panel) to the last assay chamber (FIG. 5B, right panel). The fluorescence in each assay chamber was quantified to measure dilution and this quantification is plotted along with the simulation results in FIG. 5C, which together shows a close match between simulation and experimental results.

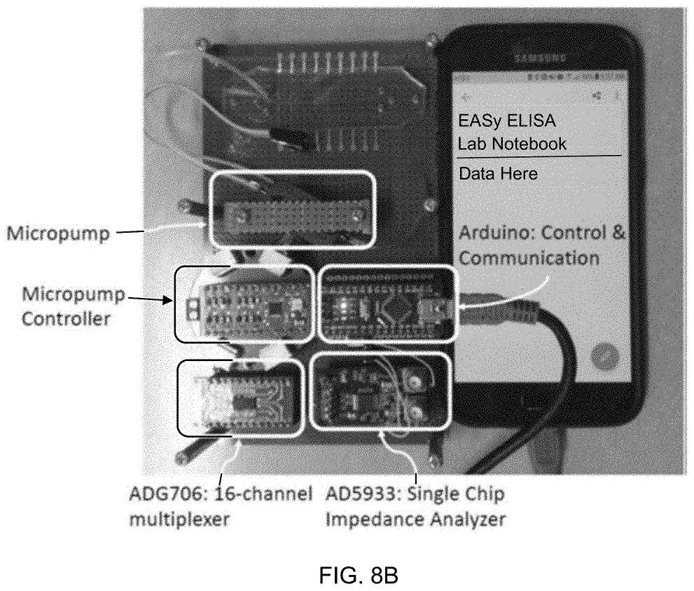

A PDMS microfluidic serial dilution network was aligned and reversibly bonded on top of the microelectrode array substrate to create the integrated EASy-ELISA chip shown in FIG. 6A. Serial dilution and electrical detection of streptavidin-HRP to biotinylated BSA was then performed using this chip. The electrical impedance measurement results for two different metallization reaction times (t=4 mins, 7.5 mins) are shown in FIG. 6B, which shows the variation of electrode impedance with target analyte concentration as estimated based on input concentration and programmed dilution factor. A limit of detection of ˜5 pM can be inferred from these results, as can be seen in FIG. 6C, which is a close-up view of a portion of FIG. 6B.

The impedance measurement results for the two different metallization times reveal another interesting feature of this overall scheme. The use of high metallization times (e.g. t=8 min) results in a sharp switch-like characteristic in the impedance versus analyte concentration curve, whereas the use of lower metallization times (e.g. t=4 min) shows a smoother shape. This feature can be exploited to tune the sensor to operate either in a ‘digital’ (i.e. ‘ON/OFF’ or threshold detection regime) or an ‘analog’ sensor regime with a linear calibration curve. These regimes are suitable for different applications. Digital detection can be used for counting assays for cells. Analog assays allow single-point quantification of biomolecules and is used for the TB diagnostic assays disclosed herein.

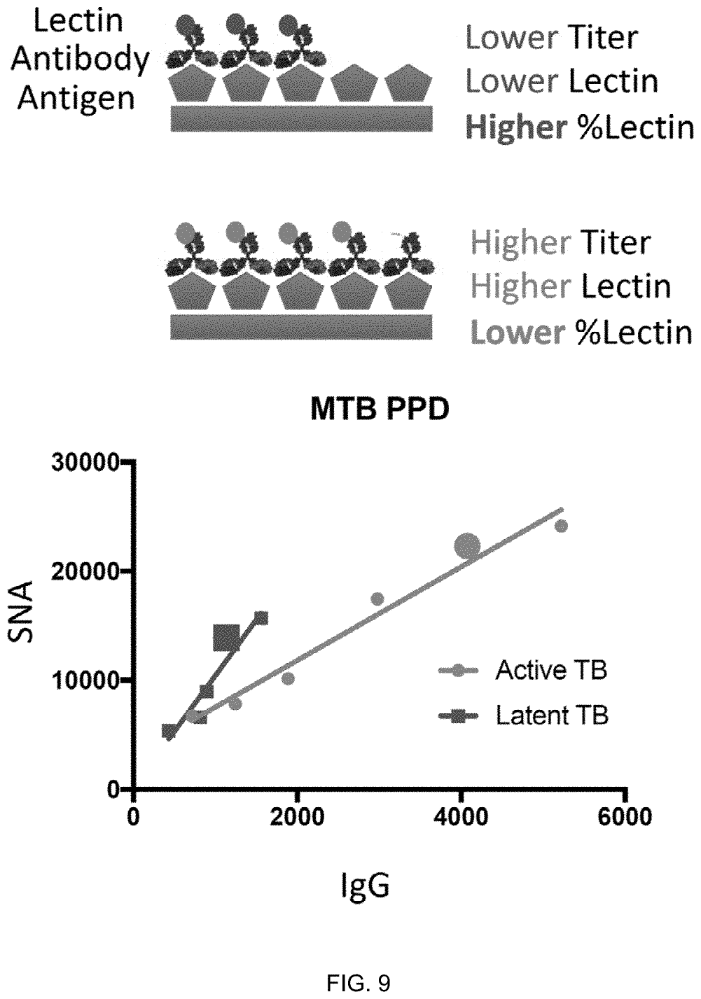

To test the system using actual samples, small volumes (e.g. ˜2-5 μL) of TB patient serum samples (n=10) with known clinical diagnoses were then analyzed using the above chip. Antibodies were captured using four different TB antigens (PPD, LAM Ag85A, CFP10) and probed with HRP-tagged anti-human-IgG antibody and two lectins (SNA, RCA1) with affinity for sialic acid and galactose, respectively, to determine a glycosylation state of the antibodies. This generated a set of impedance signatures that were then analyzed using partial least square discriminant analysis (PLS-DA) (FIGS. 7A-7C). Accurate discrimination of ATB and LTBI was obtained with unit area under the receiver-operator characteristic curve (FIG. 7C). The loading plot shows that antigen-specific antibody titers and associated lectin-binding titers both contribute to the separation between patient classes.

Point-of-Care Tuberculosis Diagnosis and Disease Stratification Using Antibody Glycan Biomarkers and Others

Tuberculosis, despite being largely curable and controllable by existing drugs, remains the world's top killer infectious disease (˜5000 deaths/day). This is at least partly due to the lack of affordable yet sensitive and specific methods for its diagnosis and stratification. Antibody detection tests, which tested for presence or absence of anti-MTB antibodies in serum and were offered in affordable dipstick formats, have earlier been found to be not sensitive and specific enough for use in TB diagnosis and have subsequently been banned by the World Health Organization (WHO). Most existing sensitive and specific diagnostic methods for TB (e.g. culture-based methods) still require significant laboratory infrastructure and technical expertise not easily available in the resource-poor settings in which the disease is endemic. Most current diagnostic methods including POC methods (e.g. Cepheid Inc.'s GeneXpert) also use sputum as a sample, which is challenging and invasive to obtain (esp. for childhood TB) and requires complex sample processing to isolate or visualize mycobacterium tuberculosis (MTB) for analysis. Further, the stratification of patients along the relatively complex spectrum of TB disease (LTBI vs. ATB) has proven challenging using existing POC methods, despite the fact that the LTBI vs. ATB distinction is critical for therapeutic decision-making. Issues such as these have led the WHO to declare the development of a rapid biomarker-based test for non-sputum samples to be a high priority need for the control and eradication of TB.

Antigen-specific antibody glycans are an interesting new class of biomarkers, which have shown potential in diagnosis and stratification of TB. They have also shown promise as biomarkers in rheumatoid arthritis, immune activation, and aging-related inflammation. Existing methods to detect and quantify these biomarkers are, however, still dependent on expensive laboratory infrastructure (e.g. mass spectrometry, capillary electrophoresis). Nevertheless, the EASy ELISA device and lectin-based antibody and antibody glycan quantitation method using the device can accurately distinguish LTBI and ATB while using only a small sample volume (e.g. a drop of blood).

Beyond these biomarkers, other TB diagnosis modalities may be ported to the EASy-ELISA platform as well. For example, the interferon gamma (IFN-g) release assay (IGRA) can aid in diagnosis of MTB infection, although it cannot differentiate LTBI and ATB. Two FDA-approved IGRAs are commercially available in the U.S.: Quantiferon-TB Gold (marketed by Qiagen) and T-Spot (marketed by Oxford Immunotec). The readout in these assays is either via an ELISA to measure IFN-g concentration or an ELISPOT assay to measure number of IFN-g secreting cells. Both of these detection modalities currently require specialized laboratory infrastructure but may be ported to the EASy-ELISA platform, e.g. using schemes such as those shown in FIGS. 1A-1C, allowing these assays to be performed as POC assays.

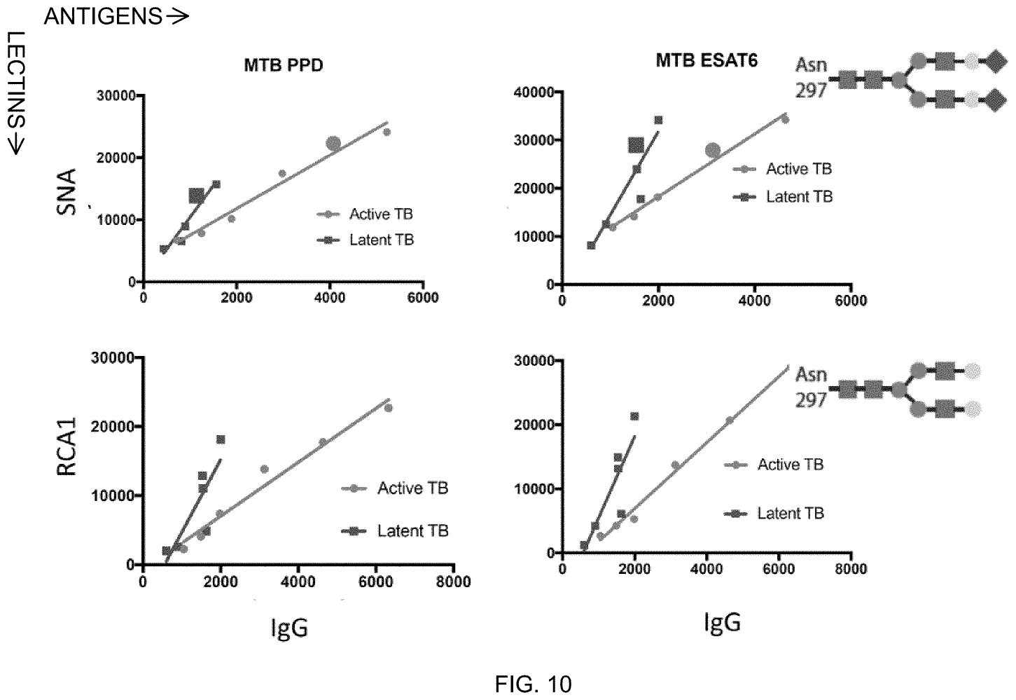

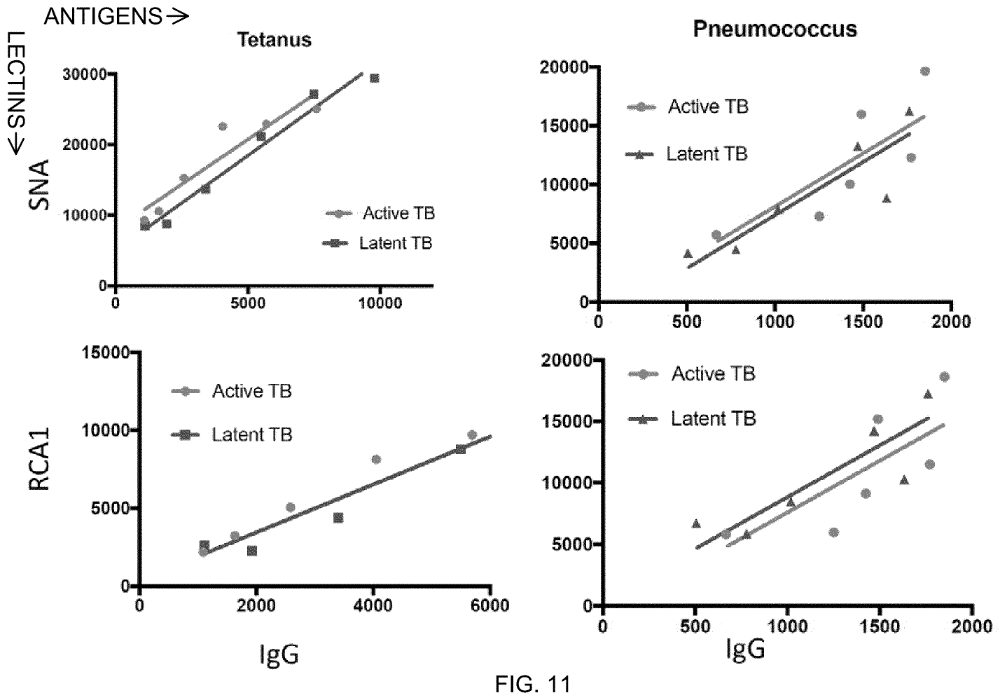

Electrical detection and integrated microfluidic sample handling facilitate the EASy-ELISA device to be developed into a commercial product, for example, as a cellphone-interfaced portable, inexpensive POC device such as that shown schematically in FIG. 8A. Such a device can serve the large market for TB diagnosis and stratification around the world where currently around 150 million TB diagnostic tests are ordered per year, including 80 million POC tests. In one particular embodiment, a cellphone/smartphone-compatible device may include a self-contained cartridge for obtaining and processing a sample (e.g. a blood sample as shown in FIG. 8A), where the cartridge then transmits data (e.g. wirelessly) regarding the test results to the cellphone or smartphone, or to another computer system or network, for processing and/or recording.

One particular embodiment of a cellphone-based device for implementing EASy-ELISA detection is shown in FIG. 8B. Shown in FIG. 8B are a smartphone that is interfaced with a circuit board to which are attached components that are needed for sample handling and data collection, including a micropump, a micropump controller, an impedance analyzer (e.g. AD5933 from Analog Devices), a multiplexer (e.g. ADG706 from Analog Devices), and a communication unit (e.g. an Arduino USB board).

Diagnosing Diseases or Conditions

Tuberculosis

In various embodiments, the methods, apparatus, and systems disclosed herein may be used to diagnose and treat a disease or condition in a subject such as a human patient. The methods may include assaying a sample obtained from the subject to determine an antibody glycosylation state, where the antibody glycosylation state is indicative of the disease or condition. If the antibody glycosylation state is indicative of the presence of the disease or condition, the method may include administering a treatment for the disease or condition.

As disclosed herein, in certain embodiments the disease or condition may be tuberculosis (TB) and in particular embodiments, the TB may be active TB. As disclosed above, various antigens may be used to detect TB, including one or more of PPD, LAM, CFP10, ESAT6, or Ag85A, and antibodies associated with active TB (vs. latent TB) may be identified based on the antibodies' glycosylation state, such as a presence or absence of sialic acid or galactose attached to the antibodies (e.g. in the Fc region of the antibodies). A subject having TB antibodies with a glycosylation state that indicates that the subject may have active TB may receive treatment based on this information. In addition, a prediction regarding the subject's disease outcome may be performed based on the glycosylation state information. Various methods may be used to determine the glycosylation state of the antibodies, including capillary electrophoresis, conventional ELISA assays, and/or EASy-ELISA technology as disclosed herein. Samples (e.g. bodily fluids) may be obtained from the subject at various regular or non-regular intervals (e.g. daily/weekly/monthly etc.) and analyzed to determine the glycosylation state and to use this information to provide diagnosis, prediction, and/or treatment for the subject.

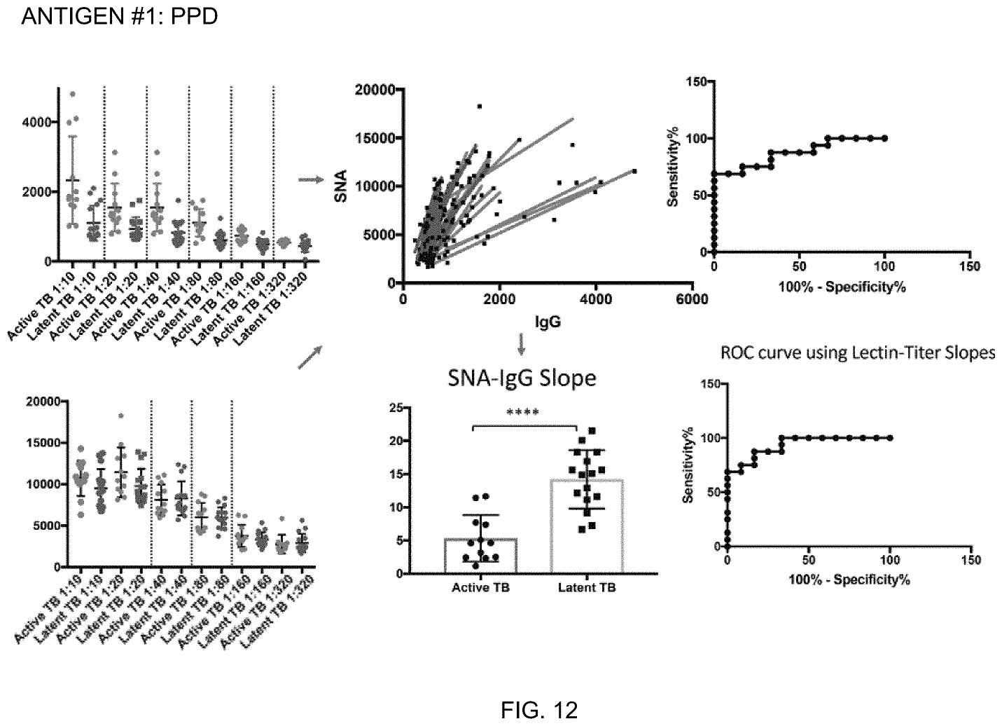

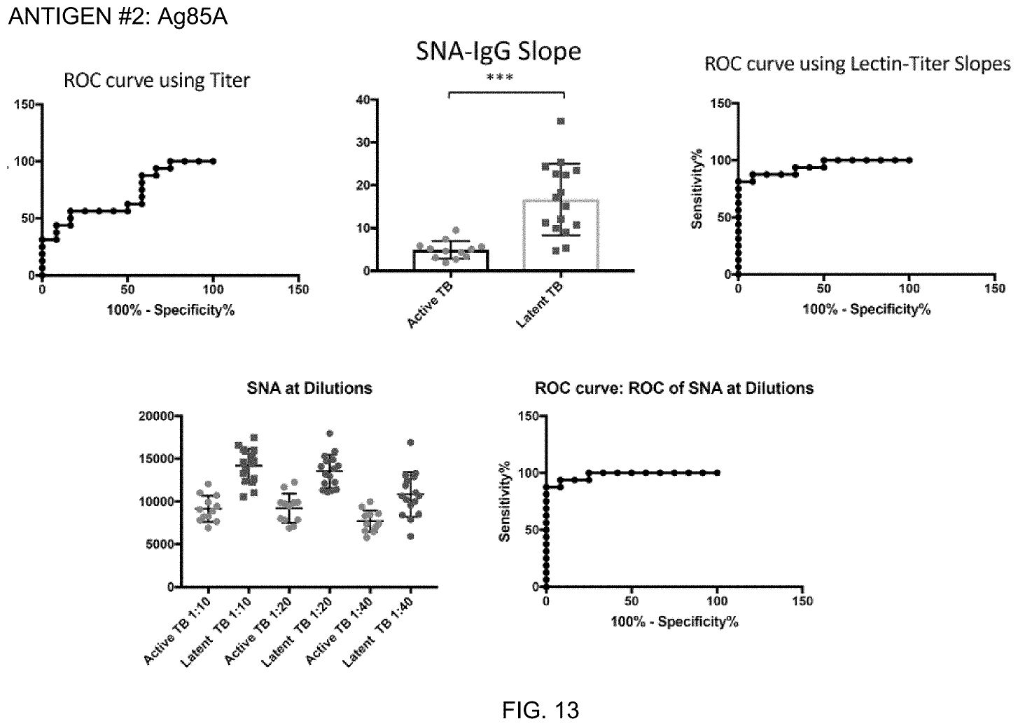

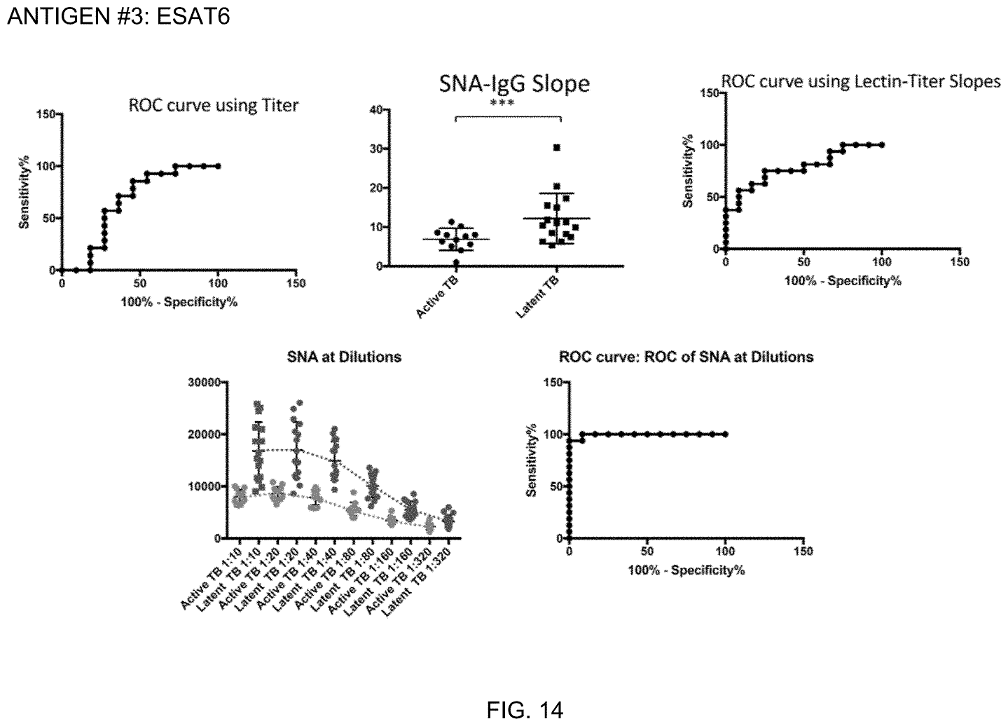

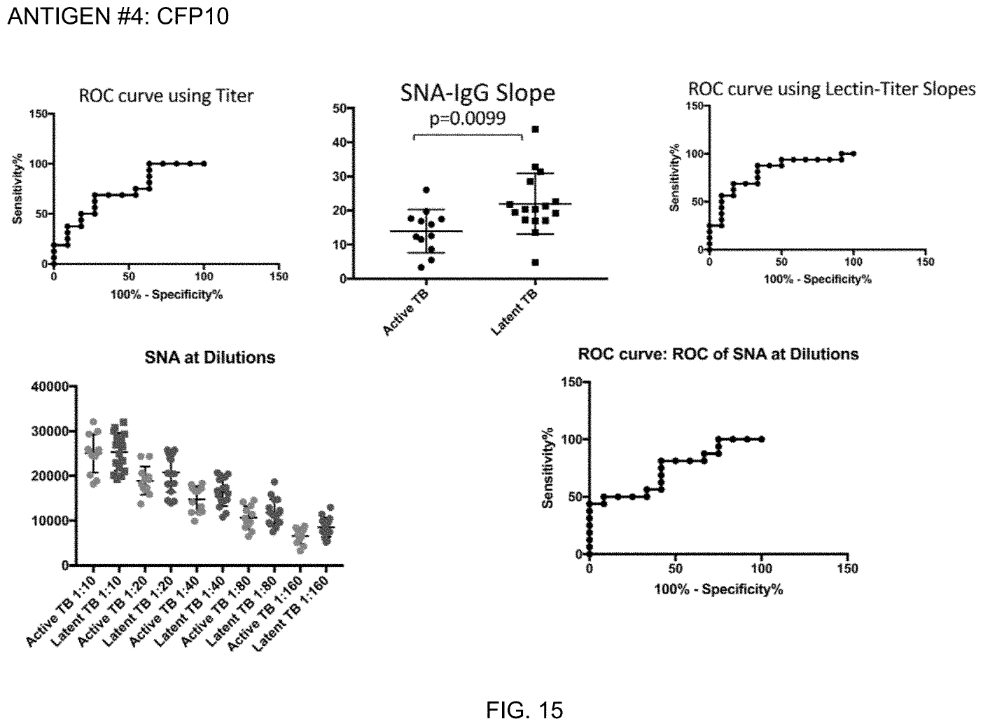

Studies in which dual dilution curves have been generated show distinct slopes of curves associated with active TB antibodies compared to latent TB antibodies. FIG. 9 shows graphs of dual dilution curves which distinguish differences in titer and lectin binding via slopes of the curves. In these graphs, serial dilutions of the samples (which include either active or latent antibodies) are probed with anti-IgG or lectin (SNA or RCA1) and the results graphed relative to one another. FIG. 10 shows dual dilution curves such as those in FIG. 9 using MTB PPD (left) or MTB ESAT6 (right) and lectins SNA (top) or RCA1 (bottom), again showing that the slopes associated with active vs. latent TB samples are different. On the other hand, graphs obtained using non-MTB antigens on active or latent TB samples, which are probed with anti-IgG and lectins, do not have different slopes: FIG. 11 shows dual dilution curves for non-MTB antigens tetanus (left) or pneumococcus (right) and lectins SNA (top) or RCA1 (bottom). FIGS. 12-15 show antigen-specific lectin-binding signatures in TB in which optimization of antigen choice (determined in part using AuROC analysis) can provide improved diagnostic power. Data are shown for antigens PPD (FIG. 12), Ag85A (FIG. 13), ESAT6 (FIG. 14), and CFP10 (FIG. 15).

Thus, the data disclosed herein, particularly in FIGS. 9-15, indicate that SNA/IgG dilution curve slopes can provide an indication of glycosylation state independent of antibody titer. In addition, SNA (lectin)/IgG slopes are significantly different for various MTB antigens tested, including PPD, Ag85A, ESAT6, and CFP10. On the other hand, SNA (lectin)/IgG slopes are not different for non-MTB-specific antigens. Finally, results of AuROC analysis of antigen-specific lectin-binding signatures in TB indicate:

[SNA/PPD slope˜SNA/Ag85A slope]>[SNA/ESAT6 slope˜SNA-CFP10 slope], and

SNA on ESAT6 and SNA on Ag85A are best classifiers (>0.98) without the use of a slope.

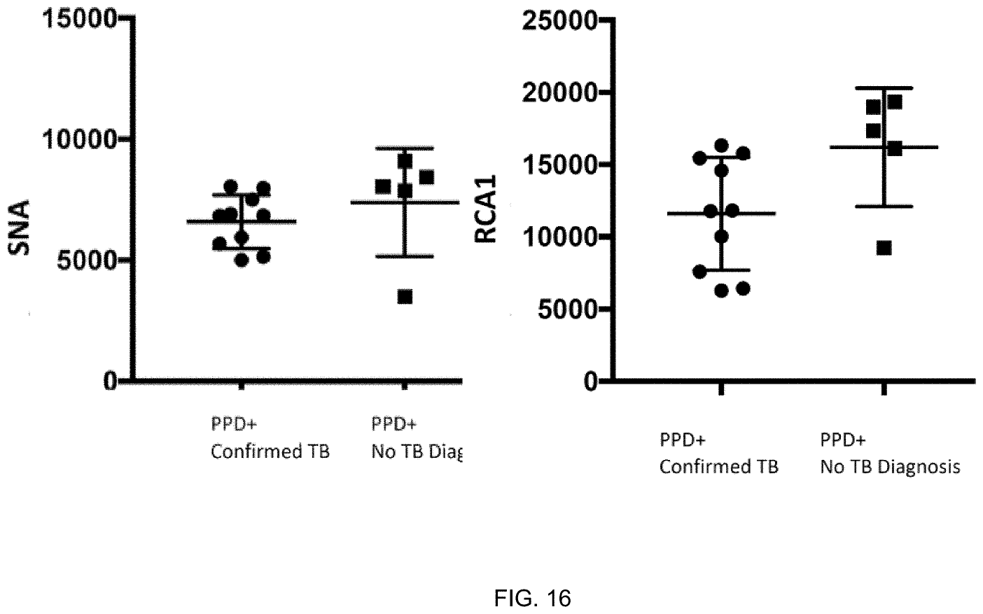

Further validation of the disclosed procedures is provided by analysis of pediatric samples from children who are PPD+ (FIG. 16). Analysis of pediatric TB samples using lectins SNA (left) or RCA1 (right) shows a difference in lectin levels in samples with confirmed TB vs. no TB diagnosis. Thus, EASy-ELISA lectin signatures can distinguish active TB in children who have PPD+ titer, which is important given that sputum extraction is especially difficult in children and given that no other blood-based diagnostic is available for TB.

Typhoid

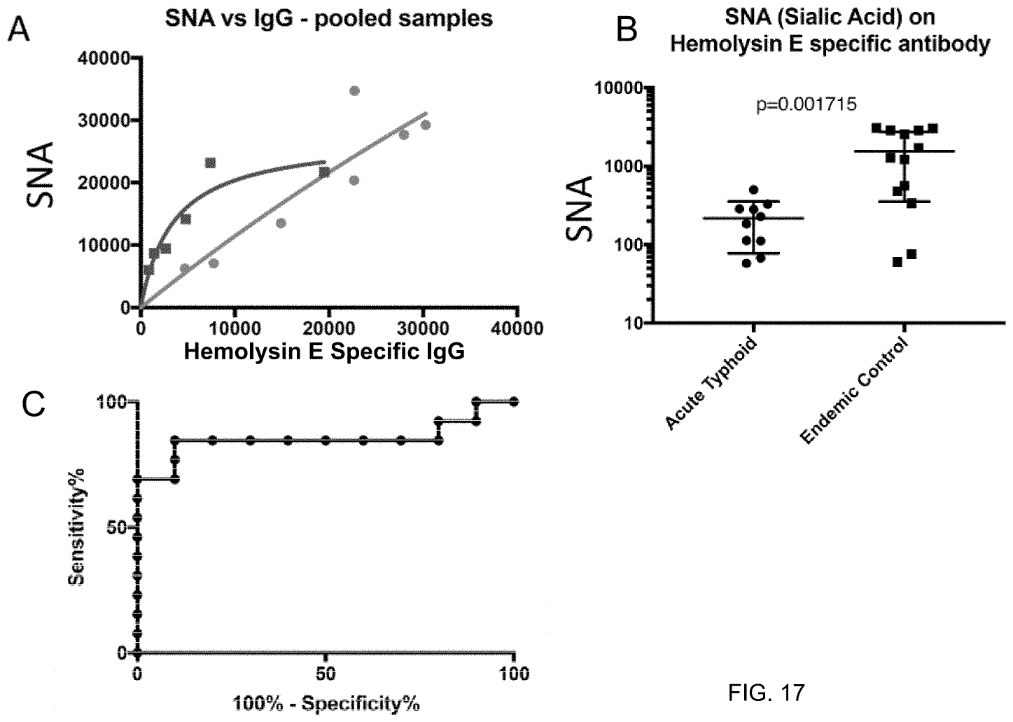

Additional experiments have shown that the above analysis is applicable to other infectious diseases, specifically to typhoid. FIG. 17 shows analysis of typhoid samples using hemolysin E-specific IgG antibodies, showing that SNA lectin binding to hemolysin E-specific antibodies captured from pooled serum samples of acute typhoid patients and healthy control adults, where both groups of samples were obtained from endemic areas (from Bangladesh). Panel A shows that significant differences in SNA binding affinity are observed, which indicates a difference in sialic acid content of these antibodies. Panel B shows SNA lectin binding to hemolysin E-specific antibodies from individual serum samples of acute typhoid patients and healthy endemic control adults. Panel C shows RoC curve analysis of the SNA-binding data shown in panel B.

Further information regarding methods, apparatus, and systems of treating, diagnosing, and/or prognosing a disease in a subject, particularly relating to detection of the glycosylation state of the antibodies present in the subject, may be found in U.S. application Ser. No. 15/520,432, which is incorporated herein by reference in its entirety.

Thus, while the invention has been described above in connection with particular embodiments and examples, the invention is not necessarily so limited, and that numerous other embodiments, examples, uses, modifications and departures from the embodiments, examples and uses are intended to be encompassed by the claims attached hereto.

1. A method for detection of antibodies in a biological sample, the method comprising:
immobilizing antigens specific to the antibodies between at least two electrodes;
binding the antibodies from the biological sample to the antigens;
binding probes linked with an enzyme to the antibodies;
exposing the enzyme to a metal substrate;
depositing a metal layer based on exposing the enzyme to the metal substrate;
measuring an electrical property of the metal layer between a first electrode of the at least two electrodes and a second electrode of the at least two electrodes; and
detecting, based on measuring the electrical property of the metal layer, the antibodies in the biological sample.

2. The method of claim 1, further comprising:
diluting a portion of the biological sample to produce a diluted biological sample;
immobilizing second antigens specific to the antibodies between at least two other electrodes;
binding the antibodies from the diluted biological sample to the second antigens;
binding second probes linked with the enzyme to the antibodies;
exposing the enzyme to the metal substrate;
depositing a second metal layer based on exposing the enzyme to the metal substrate;
measuring an electrical property of the second metal layer between a third electrode of the at least two other electrodes and a fourth electrode of the at least two other electrodes; and
detecting, based on measuring the electrical property of the second metal layer, the antibodies in the diluted biological sample.

3. The method of claim 1, wherein the detection of the antibodies is indicative of at least one of a disease state or a presence or activity of an infectious agent.

4. The method of claim 1, wherein detecting the antibodies comprises detecting a modification of the antibodies, and wherein detecting the modification of the antibodies indicates a glycosylation state.

5. The method of claim 4, wherein the glycosylation state comprises a presence or an absence of sialic acid or galactose.

6-7. (canceled)

8. The method of claim 2, wherein at least one of the probes or the second probes comprises anti-human immunoglobulin.

9. The method of claim 2, wherein at least one of the probes or the second probes comprises an agent that recognizes a glycosylation state of the antibodies.

10. The method of claim 9, wherein the agent that recognizes the glycosylation state is a lectin.

11-15. (canceled)

16. The method of claim 1, wherein the antigens are derived from or related to an infectious agent.

17-33. (canceled)

34. A serial auto-dilution device comprising:
a first inlet for a biological sample;
a first channel connected to the first inlet,
the first channel including a plurality of chambers;
a second channel connected to a source of a dilution buffer;
a first plurality of connection channels connecting the second channel to the first channel between each of the respective plurality of chambers;
a third channel connected to an outlet; and
a second plurality of connection channels connecting the first channel to the third channel between each of the respective plurality of chambers,
each of the first channel, the second channel, the third channel, the first plurality of channels, and the second plurality of channels being configured such that the biological sample flows through the first channel and the dilution buffer flows through the second channel and the first plurality of channels to produce increasingly diluted mixtures of biological sample and dilution buffer in each of the plurality of chambers.

35. (canceled)

36. The device of claim 34, wherein the first channel, the second channel, the third channel, the first plurality of channels, and the second plurality of channels are configured to generate flow of the biological sample and the dilution buffer using at least one of gravity or pressure.

37. The device of claim 34, wherein the first channel, the second channel, the third channel, the first plurality of channels, and the second plurality of channels comprise microfluidic channels.

38. The device of claim 37, further comprising:
an interdigitated microelectrode array,
wherein the plurality of chambers are connected to wells between electrodes of the interdigitated microelectrode array, and
wherein the wells include antigens specific to antibodies to be detected in the biological sample,
wherein the antigens are configured to bind to the antibodies from the biological sample;
a first mechanism configured to introduce probes linked with an enzyme,
wherein the probes are configured to bind to the antibodies or to a modification to the antibodies in the wells;
a second mechanism configured to add a metal substrate to deposit metal layers via a reaction of the enzyme in the wells;
a power source configured to generate electrical currents across electrodes of the interdigitated microelectrode array via the metal layers deposited in the wells;
a sensor configured to measure at least one property of the electrical currents; and
a processor configured to detect, based on measuring the at least one property, the antibodies or the modification to the antibodies in the biological sample.

39-53. (canceled)

54. The device of claim 37, further comprising:
an interdigitated microelectrode array,
wherein the plurality of chambers are connected to wells between electrodes of the interdigitated microelectrode array, and
wherein the wells include antibodies specific to a target to be detected in the biological sample,
wherein the antibodies are configured to bind to the target from the biological sample;
a first mechanism configured to introduce probes linked with an enzyme,
wherein the probes are configured to bind to the target in the wells;
a second mechanism configured to add a metal substrate to deposit metal layers via a reaction of the enzyme in the wells;
a power source configured to generate electrical currents across electrodes of the interdigitated microelectrode array via the metal layers deposited in the wells;
a sensor configured to measure at least one property of the electrical currents; and
a processor configured to detect, based on measuring the at least one property, the target in the biological sample.

55-63. (canceled)

64. The device of claim 38, wherein at least one of the first mechanism or the second mechanism includes an automated delivery mechanism.

65. The device of claim 64, wherein the automated delivery mechanism includes a cartridge.

66-90. (canceled)

91. A method of diagnosing a disease or condition in a subject, comprising:
assaying a sample obtained from the subject to determine an antibody glycosylation state,
the antibody glycosylation state being indicative of the disease or condition; and
diagnosing the disease or condition in the subject based on the presence of an antibody glycosylation state indicative of the disease or condition.

92. The method of claim 91, wherein the disease or condition comprises an infectious disease comprising tuberculosis (TB).

93. (canceled)

94. The method of claim 92, wherein the TB is active TB.

95. The method of claim 94, wherein the antibody glycosylation state is determined for antibodies specific to an antigen, the antigen comprising a TB antigen.

96-106. (canceled)
